(19) United States
(12) Patent Application Publication
(10) Pub. No.: US 2008/0275084 A1
(43) Pub. Date: Nov. 6, 2008

(54) Piperidines for the Treatment of Chemokine Mediated Diseases

(75) Inventors: Matthew Perry, Leicestershire (GB); Brian Springthorpe, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/915,460

(22) PCT Filed: May 24, 2006
(86) PCT No.: PCT/SE2006/000611

§ 371 Date: Nov. 26, 2007

(30) Foreign Application Priority Data
May 27, 2005 (SE) 0501212-5

(51) Int. Cl. A61K 31/4545; C07D 401/06

(52) U.S. Cl. 514/316; 546/188

(57) ABSTRACT

The present invention provides a compound of a formula (I): wherein the variables are defined herein; to a process for preparing such a compound; and to the use of such a compound in the treatment of a chemokine (such as CCR3) or H1 mediated disease state.

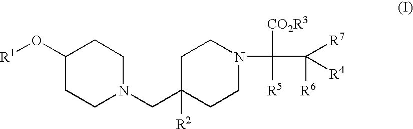

The present invention concerns piperidine derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in WO 2004/087659.

Histamine is a basic amine, 2-(4-imidazolyl)-ethylamine, and is formed from histidine by histidine decarboxylase. It is found in most tissues of the body, but is present in high concentrations in the lung, skin and in the gastrointestinal tract. At the cellular level inflammatory cells such as mast cells and basophils store large amounts of histamine. It is recognised that the degranulation of mast cells and basophils and the subsequent release of histamine is a fundamental mechanism responsible for the clinical manifestation of an allergic process. Histamine produces its actions by an effect on specific histamine G-protein coupled receptors, which are of four main types, H1, H2, H3, and H4. Histamine H1 antagonists comprise the largest class of medications used in the treatment of patients with allergic disorders, for example rhinitis or urticaria. H1 antagonists are useful in controlling the allergic response by for example blocking the action of histamine on post-capillary venule smooth muscle, resulting in decreased vascular permeability, exudation and oedema. The antagonists also produce blockade of the actions of histamine on the H1 receptors on c-type nociceptive nerve fibres, resulting in decreased itching and sneezing.

Chemokines are chemotactic cytokines that are released by a wide variety of cells to attract macrophages, T cells, eosinophils, basophils and neutrophils to sites of inflammation and also play a role in the maturation of cells of the immune system. Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C-X-C, or α) and Cys-Cys (C-C, or β) families. These are distinguished on the basis of a single amino acid insertion between the NH-proximal pair of cysteine residues and sequence similarity.

The C-X-C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

Viral infections are known to cause lung inflammation. It has been shown experimentally that the common cold increases mucosal output of eotaxin in the airways. Instillation of eotaxin into the nose can mimic some of the signs and symptoms of a common cold. (See, Greiff L et al Allergy (1999) 54(11) 1204-8 [Experimental common cold increase mucosal output of eotaxin in atopic individuals] and Kawaguchi M et al Int. Arch. Allergy Immunol. (2000) 122 S1 44 [Expression of eotaxin by normal airway epithelial cells after virus A infection].)

The present invention provides a compound of formula (I):

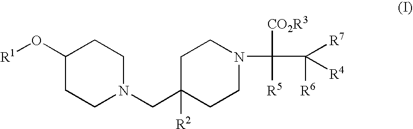

wherein:
R¹ is phenyl optionally substituted by halogen, cyano, C₁₋₄ alkyl or C₁₋₄ alkoxy;
R² is hydrogen or hydroxy;
R³ is hydrogen, C₁₋₆ alkyl or phenyl(C₁₋₄ alkyl); wherein phenyl is optionally substituted with halogen, hydroxy, nitro, S(O)_{q}(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
q is 0, 1 or 2;
R⁴ is methyl, CH(CH₃)₂, or C₃₋₇ cycloalkyl optionally substituted by C₁₋₄ alkyl;
R⁵, R⁶ and R⁷ are, independently, hydrogen or methyl;
or R⁴ and R⁵ join to form a 3-7 membered carbocyclic ring optionally substituted by C₁₋₄ alkyl; and two of the ring carbons of this ring can be joined through a 1 or 2 carbon alkylene chain (which is itself optionally substituted by C₁₋₄ alkyl) such that a bicyclic ring system is formed;
or a N-oxide thereof; or a pharmaceutically acceptable salt thereof.

Certain compounds of the present invention can exist in different isomeric forms (such as enantiomers, diastereomers, geometric isomers or tautomers). The present invention covers all such isomers and mixtures thereof in all proportions.

The compounds of the invention can be zwitterionic and all such zwitterions are within the invention.

Pharmaceutically acceptable salts include acid addition salts such as a hydrochloride, dihydrochloride, hydrobromide, phosphate, sulfate, acetate, diacetate, fumarate, maleate, malonate, succinate, tartrate, citrate, oxalate, methanesulfonate or p-toluenesulfonate.

Pharmaceutically acceptable salts also include an alkali metal (for example sodium or potassium) or alkaline earth metal (for example magnesium or calcium) salt of a compound of formula (I) wherein R³ is hydrogen. A pharmaceutically acceptable salt is, for example, a hemi-salt. In the neutral state a hemi-salt is formed by two compounds of formula (I), wherein R³ is hydrogen, and one alkaline earth metal (for example calcium).

The compounds of the invention may exist as solvates (such as hydrates) and the present invention covers all such solvates.

Halogen includes fluorine, chlorine, bromine and iodine. Halogen is, for example, fluorine or chlorine.

Alkyl is straight or branched chain and is, for example, methyl, ethyl, n-propyl, iso-propyl or tert-butyl.

Cycloalkyl is, for example, cyclopropyl, cyclopentyl or cyclohexyl.

In one particular aspect the present invention provides a compound of formula (I) wherein: R¹ is phenyl optionally substituted by halogen, cyano, C₁₋₄ alkyl or C₁₋₄ alkoxy; R² is hydrogen or hydroxy; R³ is hydrogen, C₁₋₆ alkyl or phenyl(C₁₋₄ alkyl); wherein phenyl is optionally substituted with halogen, hydroxy, nitro, S(O)_{q}(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; q is 0, 1 or 2; R⁴ is CH(CH₃)₂, or C₃₋₇ cycloalkyl optionally substituted by C₁₋₄ alkyl; R⁵ is hydrogen; R⁶ and R⁷ are both hydrogen; or R⁴ and R⁵ join to form a 3-7 membered carbocyclic ring optionally substituted by C₁₋₄ alkyl; and two of the ring carbons of this ring can be joined through a 1 or 2 carbon alkylene chain (which is itself optionally substituted by C₁₋₄ alkyl) such that a bicyclic ring system is formed; or a N-oxide thereof; or a pharmaceutically acceptable salt thereof.

In a further aspect the present invention provides a compound of formula (I) wherein R¹ is phenyl optionally substituted (for example with two or three of the same or different) with fluorine, chlorine, cyano, C₁₋₄ alkyl (for example methyl) or C₁₋₄ alkoxy (for example methoxy).

In another aspect the present invention provides a compound wherein R¹ is phenyl optionally substituted (for example with two or three of the same or different) with fluorine, chlorine, cyano or C₁₋₄ alkyl (for example methyl).

In yet another aspect the present invention provides a compound wherein R¹ is phenyl substituted by two or three substituents independently selected from: fluorine, chlorine, cyano and methyl.

In yet another aspect the present invention provides a compound wherein R¹ is phenyl substituted by two or three substituents independently selected from: fluorine, chlorine and methyl.

In a further aspect the present invention provides a compound wherein R¹ is phenyl substituted by two or three substituents independently selected from: chlorine and methyl. For example R¹ is 3,4-dichlorophenyl, 2,4-dichloro-3-methylphenyl or 3,4-dichloro-2-methylphenyl. R¹ can also be 4-chloro-2-methylphenyl or 4-fluoro-2-methylphenyl.

In a still further aspect the present invention provides a compound wherein R² is hydrogen.

In another aspect the present invention provides a compound wherein R³ is hydrogen or C₁₋₆ alkyl (for example methyl or ethyl).

In yet another aspect the present invention provides a compound wherein R³ is hydrogen.

In a further aspect the present invention provides a sodium or potassium salt of a compound of formula (I) wherein R³ is hydrogen.

In a still further aspect the present invention provides a compound wherein R⁴ is CH(CH₃)₂.

In another aspect the present invention provides a compound wherein R⁴ is C₃₋₇ cycloalkyl optionally substituted by C₁₋₄ alkyl.

In a further aspect the present invention provides a compound wherein R⁴ is C₃₋₆ cycloalkyl (for example cyclopropyl, cyclopentyl or cyclohexyl).

In a still further aspect the present invention provides a compound wherein R⁵ is hydrogen.

In a further aspect the present invention provides a compound wherein R⁵ is methyl.

In another aspect the present invention provides a compound wherein R⁴ and R⁵ join to form a 3-7 membered ring (for example a cyclohexyl or cyclopentyl ring).

In yet another aspect the present invention provides a compound wherein R¹ and R⁵ join to form a 3-7 membered ring and two of the ring carbons of this ring join through a 1 or 2 carbon alkylene chain such that a bicyclic ring system (for example a bicyclo[2.2.1]heptane ring system).

In a further aspect the present invention provides a compound wherein R⁶ and R⁷ are both hydrogen.

In another aspect the present invention provides a compound wherein R², R⁶ and R⁷ are all hydrogen; R⁵ is methyl; and R⁴ is CH(CH₃)₂.

In yet another aspect the present invention provides a compound wherein R¹ is phenyl optionally substituted by halogen (for example chloro or fluoro) or C₁₋₄ alkyl (for example methyl); R² is hydrogen; R³ is hydrogen, C₁₋₆ alkyl (for example methyl); R⁴ is methyl, CH(CH₃)₂, or C₃₋₇ cycloalkyl (for example cyclopropyl, cyclopentyl or cyclohexyl); R⁵ is hydrogen or methyl; or R⁴ and R⁵ join to form a 3-7 membered carbocyclic ring (for example cyclopentyl or cyclohexyl); R⁶ is hydrogen or methyl; and R⁷ is hydrogen.

In a still further aspect the present invention provides a compound of formula (I) wherein: R¹ is phenyl optionally substituted by halogen (for example chloro) or C₁₋₄ alkyl (for example methyl); R² is hydrogen; R³ is hydrogen or C₁₋₆ alkyl (for example methyl); R⁴ is CH(CH₃)₂, or C₃₋₇ cycloalkyl (for example cyclopropyl, cyclopentyl or cyclohexyl); R⁵ is hydrogen; or R⁴ and R⁵ join to form a 3-7 membered carbocyclic ring (for example cyclopentyl or cyclohexyl); and two of the ring carbons of this ring can be joined through a 1 or 2 carbon alkylene chain such that a bicyclic ring system (for example a bicyclo[2.2.1]heptane ring system) is formed.

A compound of formula (I) that is:
(2S)-3-Cyclohexyl-2-(4-{[4-(3,4-dichloro-2-methylphenoxy)piperidin-1-yl]methyl}piperidin-1-yl)propanoic acid;
(2S)-3-Cyclohexyl-2-(4-{[4-(3,4-dichloro-2-methylphenoxy)piperidin-1-yl]methyl}piperidin-1-yl)propanoic acid;
(2S)-3-Cyclopropyl-2-(4-{[4-(3,4-dichloro-2-methylphenoxy)piperidin-1-yl]methyl}piperidin-1-yl)propanoic acid;
(2S)-3-Cyclopentyl-2-(4-{[4-(3,4-dichlorophenoxy)piperidin-1-yl]methyl}piperidin-1-yl)propanoic acid;
3-Cyclopentyl-2-{4-[4-(3,4-dichloro-2-methyl-phenoxy)-piperidin-1-ylmethyl]-piperidin-1-yl}-propionic acid;
1-(4-{[4-(3,4-Dichlorophenoxy)piperidin-1-yl]methyl}piperidin-1-yl)cyclohexanecarboxylic acid;
1-(4-{[4-(3,4-Dichloro-2-methylphenoxy)piperidin-1-yl]methyl}piperidin-1-yl)cyclohexanecarboxylic acid;
1-(4-{[4-(3,4-Dichloro-2-methylphenoxy)piperidin-1-yl]methyl}piperidin-1-yl)cyclopentanecarboxylic acid;
(2S)-2-{4-[4-(3,4-Dichloro-2-methyl-phenoxy)-piperidin-1-ylmethyl]-piperidin-1-yl}-4-methyl-pentanoic acid;
2-{4-[4-(3,4-Dichloro-phenoxy)-piperidin-1-ylmethyl]-piperidin-1-yl}-4-methyl-pentanoic acid;
2-{4-[4-(4-Chloro-2-methyl-phenoxy)-piperidin-1-ylmethyl]-piperidin-1-yl}-3-methyl-butyric acid;
1-{4-[4-(4-Fluoro-2-methyl-phenoxy)-piperidin-1-ylmethyl]-piperidin-1-yl}-cyclohexanecarboxylic acid;
1-(4-{[4-(4-Chloro-2-methylphenoxy)piperidin-1-yl]methyl}piperidin 1-yl)cyclohexanecarboxylic acid;
(2S)-2-(4-{[4-(3,4-Dichlorophenoxy)piperidin-1-yl]methyl}piperidin-1-yl)-3-methylbutanoic acid;
(2S)-2-{4-[4-(3,4-Dichloro-2-methyl-phenoxy)-piperidin-1-ylmethyl]-piperidin-1-yl}-2,4-dimethyl-pentanoic acid; or,
(2S)-2-{4-[4-(4-Chloro-2-methyl-phenoxy)-piperidin-1-ylmethyl]-piperidin-1-yl}-2,4-dimethyl-pentanoic acid;
   or a pharmaceutically acceptable salt thereof.

The compounds of the present invention can be prepared as described below or by methods analogous to those described in WO 2004/087659.

A compound of formula (I) can be prepared by reacting a compound of formula (II):

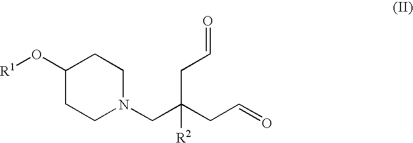

with a compound of formula (III):

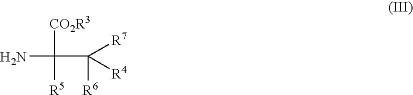

in the presence of NaBH(OAc)₃ or NaBH₃(CN) in a suitable solvent (for example an aliphatic alcohol such as methanol or ethanol) at a suitable temperature (such as in the range 0° C. to 30° C.).

Alternatively, a compound of formula (I), where R³ is alkyl or phenylalkyl, can be prepared by reacting a compound of formula (II) with a compound of formula (III), where R³ is alkyl or phenylalkyl, in the presence of NaBH(OAc)₃ in the presence of a suitable base (such as a tertiary amine, for example Hunigs base or triethylamine) in a suitable solvent (such as tetrahydrofuran) at a suitable temperature (such as in the range 0° C. to 30° C.).

For a compound of formula (I):
when R³ is hydrogen said compound may be converted to a compound of the invention where R³ is not hydrogen by a standard esterification or salt formation method well known in the art; and,
when R³ is not hydrogen said compound may be converted to a compound of the invention where R³ is hydrogen by a standard ester hydrolysis or acidification method well known in the art.
   Such methods are described in undergraduate organic chemistry textbooks (such as Advanced Organic Chemistry by J March, 5^{th} edition M B Smith and J March, Wiley, 2001).

A compound of formula (II) can be prepared by reacting a compound of formula (IV):

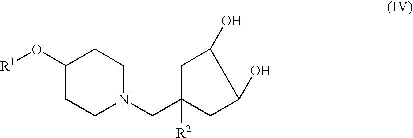

with lead tetra-acetate in the presence of sodium carbonate in dichloromethane, or by sodium periodate in water.

The preparations of various phenoxy piperidines and other intermediates are described in the literature and WO 2004/087659.

In the above processes it may be desirable or necessary to protect an acid group or a hydroxy or other potentially reactive group. Suitable protecting groups and details of processes for adding and removing such groups may be found in “Protective Groups in Organic Synthesis”, 3rd Edition (1999) by Greene and Wuts.

In another aspect the present invention provides processes for the preparation of compounds of formula (I).

The compounds of formula (I) have activity as pharmaceuticals, in particular as modulators of chemokine receptor (for example CCR3) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

Examples of these Conditions are

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthropathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymyalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthralgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Croh's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopenic purpura, eosinophilic fascitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinophilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

The compounds of formula (I) or a pharmaceutically acceptable salt thereof, are also H1 antagonists (and can, therefore, be used in the treatment of allergic disorders); and may also be used to control a sign and/or symptom of what is commonly referred to as a cold (for example a sign and/or symptom of a common cold or influenza or other associated respiratory virus infection).

According to a further feature of the present invention there is provided a method for treating a chemokine mediated disease state (for example a CCR3 mediated disease state) in a mammal, such as man, suffering from, or at risk of, said disease state, which comprises administering to a mammal in need of such treatment a therapeutically effective amount of a compound of the formula (I) or a pharmaceutically acceptable salt thereof.

According to another feature of the present invention there is provided a method for antagonising H1 in a mammal, such as man, suffering from, or at risk of, an H1 mediated disease state, which comprises administering to a mammal in need of such treatment a therapeutically effective amount of a compound of the formula (I) or a pharmaceutically acceptable salt thereof.

According to yet another feature of the present invention there is provided a method for treating a sign and/or symptom of what is commonly referred to as a cold in a mammal, such as man, suffering from, or at risk of, said disease state, which comprises administering to a mammal in need of such treatment a therapeutically effective amount of a compound of the formula (I) or a pharmaceutically acceptable salt thereof.

The invention also provides a compound of the formula (I), or a pharmaceutically acceptable salt thereof, for use in therapy.

In another aspect the invention provides the use of a compound of formula (I), or a pharmaceutically acceptable salt thereof, in the manufacture of a medicament for use in therapy (for example modulating chemokine receptor activity (for example CCR3 receptor activity), antagonising H1 or treating a sign and/or symptom of what is commonly referred to as a cold).

The invention further provides the use of a compound of formula (I), or a pharmaceutically acceptable salt thereof, in the manufacture of a medicament for use in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthropathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymyalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthralgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis, iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinophilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema;
in a mammal (for example man).

In a further aspect the invention provides a compound of formula (I), or a pharmaceutically acceptable salt thereof, for use in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulent, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

In a still further aspect a compound of formula (I), or a pharmaceutically acceptable salt thereof, is useful in the treatment of asthma.

The present invention also provides the use of a compound of formula (I), or a pharmaceutically acceptable salt thereof, in the manufacture of a medicament for use in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulent, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

The present invention also provides the use of a compound of formula (I), or a pharmaceutically acceptable salt thereof, in the manufacture of a medicament for use in the treatment of an infection due to respiratory syncytial virus.

In order to use a compound of the invention, or a pharmaceutically acceptable salt thereof, for the therapeutic treatment of a mammal, such as man, said ingredient is normally formulated in accordance with standard pharmaceutical practice as a pharmaceutical composition. Therefore in another aspect the present invention provides a pharmaceutical composition which comprises a compound of the formula (I), or a pharmaceutically acceptable salt thereof (active ingredient), and a pharmaceutically acceptable adjuvant, diluent or carrier.

In a further aspect the present invention provides a process for the preparation of said composition which comprises mixing active ingredient with a pharmaceutically acceptable adjuvant, diluent or carrier. Depending on the mode of administration, the pharmaceutical composition will, for example, comprise from 0.05 to 99% w (percent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art. A suitable pharmaceutical composition of this invention is one suitable for oral administration in unit dosage form, for example a tablet or capsule which contains between 0.1 mg and 1 g of active ingredient.

Each patient may receive, for example, a dose of 0.01 mgkg⁻¹ to 100 mgkg⁻¹, for example in the range of 0.1 mgkg⁻¹ to 20 mgkg⁻¹, of the active ingredient administered, for example, 1 to 4 times per day.

The invention further relates to a combination therapy wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; for example collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-yls such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agent including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfmavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptiline or other anti-depressant agents, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of p38; (xxv) agent modulating the function of Toll-like receptors (TLR), (xxvi) agent modulating the activity of purinergic receptors such as P2X7; (xxvii) inhibitor of transcription factor activation such as NFkB, API, or STATS; or (xxviii) a non-steroidal glucocorticoid receptor agonist.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antiestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erb b2 antibody trastuzumab, or the anti-erb b1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or,
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be illustrated by the following non-limiting examples in which, unless stated otherwise:

(i) when given, ¹H NMR data is quoted and is in the form of delta values for major diagnostic protons, given in parts per million (ppm) relative to tetramethylsilane (TMS) as an internal standard, determined at 300 MHz or 400 MHz using perdeuterio DMSO-D6 (CD₃SOCD₃) or CDCl₃ as the solvent unless otherwise stated;
(ii) mass spectra (MS) were run with an electron energy of 70 electron volts in the chemical ionisation (CI) mode using a direct exposure probe; where indicated ionisation was effected by electron impact (EI) or fast atom bombardment (FAB); where values for m/z are given, generally only ions which indicate the parent mass are reported, and unless otherwise stated the mass ion quoted is the positive mass ion -(M+H)⁺;
(iii) the title and sub-title compounds of the examples and methods were named using the index name program from Advanced Chemistry Development Inc, version 6.00, or with the AUTONOM program available from Beilstein informations systeme GmbH;
(iv) unless stated otherwise, reverse phase HPLC was conducted using a “Symmetry”, “NovaPak” or “Xterra” reverse phase silica column, all available from Waters Corp.;
(v) for analytical HPLC the following conditions were used:
Reverse phase analytical HPLC (Hewlett Packard Series 1100) using Waters “Symmetry” C8 column 3.5 μm; 4.6×50 mm column using 0.1% ammonium acetate/acetonitrile gradients at 2 mL/min given as % aqueous
STANDARD 75% to 5% over 3 min
FAST 45% to 5% over 2.5 min

### MEDIUM FAST 65% to 5% in 2.5 min

### SLOW 95% to 50% in 2.5 min

SUPERSLOW 100% to 80% in 2.5 min; and
(vi) the following abbreviations are used:

| | |
|---|---|
| RPHPLC | Reverse phase high pressure liquid chromatography |
| min | minutes |
| h | hour |

### EXAMPLE 1

This Example illustrates the preparation of methyl (2S)-3-cyclohexyl-2-(4-{[4-(3,4-dichlorophenoxy)piperidin-1-yl]methyl}piperidin-1-yl)propanoate

4-[[4-(3,4-Dichlorophenoxy)-1-piperidinyl]methyl]-1,2-cyclopentanediol (WO200487659; 380 mg) was dissolved in water (5 mL) containing 1 drop acetic acid. Sodium periodate (229 mg) was added and the mixture was stirred for 1 h. Potassium carbonate (190 mg) was added and the mixture was extracted with dichloromethane (2×10 mL). The organic phases were dried and filtered and the resulting solution was added to a solution of methyl 3-cyclohexyl-L-alaninate hydrochloride (234 mg), sodium triacetoxyborohydride (513 mg), triethylamine (0.16 mL) and acetic acid (0.1 mL) in dichloromethane (10 mL). The mixture was stirred for 2.5 h and was then poured into aqueous sodium bicarbonate solution. The mixture was extracted with ethyl acetate; the organic phase was dried, filtered and evaporated. The residue was purified by chromatography (silica, eluent ethyl acetate) to give the title compound (253 mg).

MS [M+H]⁺ (ES+) 511/513; Retention time 2.99 fast gradient.

The following compounds were prepared analogously from the appropriate esters and diols:

| | | MS | Retention |
|---|---|---|---|
| | | [M + H]⁺ | time |
| Example | Name | (ES+) | gradient |
| 2 | Methyl (2S)-3-cyclohexyl-2-(4-{[4-(3,4-dichloro-2- | | 3.6 (fast) |
| | methylphenoxy)piperidin-1-yl]methyl}piperidin-1- | | |
| | yl)propanoate | | |
| 3 | Methyl (2S)-3-cyclopropyl-2-(4-{[4-(3,4-dichloro-2- | 483/485 | 2.19 (fast) |
| | methylphenoxy)piperidin-1-yl]methyl}piperidin-1- | | |
| | yl)propanoate | | |
| 4 | Methyl (2S)-3-cyclopentyl-2-(4-{[4-(3,4- | 497/499 | 2.70 (fast) |
| | dichlorophenoxy)piperidin-1-yl]methyl}piperidin-1- | | |
| | yl)propanoate | | |
| 5 | Methyl (2S)-3-cyclopentyl-2-(4-{[4-(3,4-dichloro-2- | 511/513 | 3.04 (fast) |
| | methylphenoxy)piperidin-1-yl]methyl}piperidin-1- | | |
| | yl)propanoate | | |
| 6 | Methyl 1-(4-{[4-(3,4-dichlorophenoxy)piperidin-1- | 483/485 | 2.61 (fast) |
| | yl]methyl}piperidin-1-yl)cyclohexanecarboxylate | | |
| 7 | Methyl 1-(4-{[4-(3,4-dichloro-2- | 497/499 | 2.19 (fast) |
| | methylphenoxy)piperidin-1-yl]methyl}piperidin-1- | | |
| | yl)cyclohexanecarboxylate | | |
| 8 | Methyl 1-(4-{[4-(3,4-dichloro-2- | 483/485 | 2.19 (fast) |
| | methylphenoxy)piperidin-1-yl]methyl}piperidin-1- | | |
| | yl)cyclopentanecarboxylate | | |
| 9 | Methyl (2S)-2-{4-[4-(3,4-Dichloro-2-methyl- | 485/487 | 2.57 (fast) |
| | phenoxy)-piperidin-1-ylmethyl]-piperidin-1-yl}-4- | | |
| | methyl-pentanoate | | |
| 10 | Methyl (2S)-2-{4-[4-(3,4-Dichloro-phenoxy)- | 471/473 | 2.39 (fast) |
| | piperidin-1-ylmethyl]-piperidin-1-yl}-4-methyl- | | |
| | pentanoate | | |
| 11 | Methyl (2S)-2-{4-[4-(4-Chloro-2-methyl-phenoxy)- | | |
| | piperidin-1-ylmethyl]-piperidin-1-yl}-3-methyl- | | |
| | butyrate | | |
| 12 | tert-Butyl 1-(4-{[4-(4-fluoro-2- | 489 | 2.86 (fast) |
| | methylphenoxy)piperidin-1-yl]methyl}piperidin-1- | | |
| | yl)cyclohexanecarboxylate | | |
| 13 | tert-Butyl 1-(4-{[4-(4-chloro-2- | 505/507 | 3.45 (fast) |
| | methylphenoxy)piperidin-1-yl]methyl}piperidin-1- | | |
| | yl)cyclohexanecarboxylate | | |
| 14 | Methyl (2S)-2-(4-{[4-(3,4- | 457/459 | |
| | dichlorophenoxy)piperidin-1-yl]methyl}piperidin-1- | | |
| | yl)-3-methylbutanoate | | |
| 15 | Methyl (2S) 2-{4-[4-(3,4-dichloro-2-methyl- | 499/501 | 3.20 (fast) |
| | phenoxy)-piperidin-1-ylmethyl]-piperidin-1-yl}-2,4- | | |
| | dimethyl-pentanoate | | |
| 16 | Methyl (2S) 2-{4-[4-(4-chloro-2-methyl-phenoxy)- | | 2.52 (fast) |
| | piperidin-1-ylmethyl]-piperidin-1-yl}-2,4-dimethyl- | | |
| | pentanoate | | |

### EXAMPLE 1A

This Example illustrates the preparation of (2S)-3-cyclohexyl-2-(4-{[4-(3,4-dichloro-2-methylphenoxy)piperidin-1-yl]methyl}piperidin-1-yl)propanoic acid

Methyl (2S)-3-cyclohexyl-2-(4-{[4-(3,4-dichlorophenoxy)piperidin-1-yl]methyl}piperidin-1-yl)propanoate (253 mg; Example 1) was dissolved in tetrahydrofuran (15 mL) and a solution of lithium hydroxide (170 mg) in water (10 μL) was added. The mixture was stirred overnight and then the volatiles were evaporated. The residue was acidified with acetic acid and purified by RPHPLC (gradient 75:25→5:95 0.1% aq ammonium acetate:acetonitrile with loading via a 7.5% acetonitrile at-column dilution stream) to give the title compound (28 mg).

¹H NMR δ_{(CD3OD+NaOD)} 0.81-1.07 (4H, m), 1.11-1.42 (8H, m), 1.50-1.84 (8H, m), 1.89-2.08 (2H, m), 2.23 (2H, d), 2.28-2.40 (4H, m), 2.65-2.78 (2H, m), 2.93-3.12 (3H, m), 4.34-4.46 (1H, m), 6.90 (1H, dd), 7.10 (1H, d), 7.39 (1H, d)

MS (ES-ve) (M−H)− 495/497

The following compounds were prepared from the corresponding ester using the method of Example 1A:

| | | MS [M + H]⁺ | |
|---|---|---|---|
| | | (APCI+); | ¹H NMR δ_{(CD3OD+NaOD)} |
| Example | Name | RT (std) | (unless otherwise indicated) |
| 2A | (2S)-3-Cyclohexyl-2-(4-{[4- | 511/513 | δ_{(D2O)} 0.85-1.43 (7H, m), |
| | (3,4-dichloro-2- | | 1.57-1.87 (8H, m), |
| | methylphenoxy)piperidin-1- | | 1.96-2.42 (10H, m), 2.97-3.37 (6H, m), |
| | yl]methyl}piperidin-1- | | 3.51-3.79 (5H, m), |
| | yl)propanoic acid | | 4.55-4.66 (0.5H, m), |
| | | | 4.82-4.86 (0.5H, m), 6.99 (0.5H, d), |
| | | | 7.05 (0.5H, d), 7.40 (1H, d) |
| 3A | (2S)-3-Cyclopropyl-2-(4-{[4- | 469/471 | −0.03-0.03 (1H, m), |
| | (3,4-dichloro-2- | | 0.06-0.15 (1H, m), 0.39 (2H, d), |
| | methylphenoxy)piperidin-1- | | 0.63-0.76 (1H, m), |
| | yl]methyl}piperidin-1- | | 1.08-1.32 (3H, m), 1.46-1.58 (1H, |
| | yl)propanoic acid | | m), 1.66-1.83 (5H, m), |
| | | | 1.92-2.02 (2H, m), 2.19 (2H, d), |
| | | | 2.21-2.37 (4H, m), 2.27 (3H, |
| | | | s), 2.57-2.69 (2H, m), |
| | | | 2.90-3.01 (3H, m), 4.35-4.43 (1H, |
| | | | m), 6.88 (1H, d), 7.24 (1H, d) |
| 4A | (2S)-3-Cyclopentyl-2-(4-{[4- | 483/485 | 1.06-1.20 (2H, m), |
| | (3,4- | | 1.22-1.34 (1H, m), 1.38-1.46 (1H, |
| | dichlorophenoxy)piperidin-1- | | m), 1.47-1.66 (5H, m), |
| | yl]methyl}piperidin-1- | | 1.69-1.84 (7H, m), 1.84-1.94 (2H, |
| | yl)propanoic acid | | m), 1.95-2.03 (2H, m), |
| | | | 2.21 (2H, d), 2.23-2.37 (4H, m), |
| | | | 2.64-2.74 (2H, m), |
| | | | 2.93-3.02 (3H, m), 4.33-4.41 (1H, |
| | | | m), 6.87 (1H, dd), 7.07 (1H, |
| | | | d), 7.36 (1H, d) |
| 5A | 3-Cyclopentyl-2-{4-[4-(3,4- | 497/499 | 1.06-1.20 (3H, m), |
| | dichloro-2-methyl-phenoxy)- | | 1.21-1.35 (1H, m), 1.38-1.66 (6H, |
| | piperidin-1-ylmethyl]- | | m), 1.70-1.84 (6H, m), |
| | piperidin-1-yl}-propionic acid | | 1.85-1.94 (2H, m), 1.95-2.03 (2H, |
| | | | m), 2.21 (2H, d), |
| | | | 2.24-2.38 (4H, m), 2.30 (3H, s), |
| | | | 2.60-2.72 (2H, m), 2.93-3.02 (3H, |
| | | | m), 4.36-4.46 (1H, m), |
| | | | 6.90 (1H, d), 7.27 (1H, d) |
| 6A | 1-(4-{[4-(3,4- | 469/471 | 1.12-1.32 (4H, m), |
| | dichlorophenoxy)piperidin-1- | 1.48 | 1.35-1.44 (2H, m), 1.48-1.56 (2H, |
| | yl]methyl}piperidin-1- | | m), 1.60-1.68 (2H, m), |
| | yl)cyclohexanecarboxylic acid | | 1.69-1.79 (4H, m), 1.94-2.02 (2H, |
| | | | m), 2.15-2.32 (7H, m), |
| | | | 2.64-2.74 (2H, m), 3.09-3.16 (2H, |
| | | | m), 4.32-4.41 (1H, m), |
| | | | 6.87 (1H, dd), 7.08 (1H, d), |
| | | | 7.36 (1H, d) |
| 7A | 1-(4-{[4-(3,4-dichloro-2- | 483/485 | 1.13-1.32 (5H, m), |
| | methylphenoxy)piperidin-1- | 0.77 (fast) | 1.35-1.44 (2H, m), 1.48-1.56 (2H, |
| | yl]methyl}piperidin-1- | | m), 1.61-1.69 (2H, m), |
| | yl)cyclohexanecarboxylic acid | | 1.70-1.84 (4H, m), 1.94-2.03 (2H, |
| | | | m), 2.15-2.37 (8H, m), |
| | | | 2.30 (3H, s), 2.62-2.70 (2H, m), |
| | | | 3.09-3.16 (2H, m), |
| | | | 4.37-4.44 (1H, m), 6.90 (1H, d), |
| | | | 7.26 (1H, d) |
| 8A | 1-(4-{[4-(3,4-dichloro-2- | 469/471 | 1.17-1.29 (2H, m), |
| | methylphenoxy)piperidin-1- | 0.72 (fast) | 1.41-1.84 (11H, m), |
| | yl]methyl}piperidin-1- | | 1.94-2.04 (2H, m), 2.21 (2H, d), |
| | yl)cyclopentanecarboxylic acid | | 2.31 (3H, s), 2.32-2.42 (6H, m), |
| | | | 2.61-2.71 (2H, m), |
| | | | 2.94-3.01 (2H, m), 4.37-4.45 (1H, |
| | | | m), 6.90 (1H, d), 7.26 (1H, d) |
| 9A | (2S)-2-{4-[4-(3,4-Dichloro-2- | 469/471 | 0.92 (3H, d), 0.94 (3H, d), |
| | methyl-phenoxy)-piperidin-1- | (APCI−) | 1.11-1.22 (1H, m), |
| | ylmethyl]-piperidin-1-yl}-4- | | 1.22-1.35 (2H, m), 1.49-1.66 (2H, m), |
| | methyl-pentanoic acid | | 1.70-1.84 (5H, m), |
| | | | 1.94-2.03 (2H, m), 2.21 (2H, d), |
| | | | 2.24-2.37 (4H, m), 2.30 (3H, |
| | | | s), 2.61-2.70 (2H, m), |
| | | | 2.94-3.03 (3H, m), 4.37-4.45 (1H, |
| | | | m), 6.90 (1H, d), 7.25 (1H, d) |
| 10A | 2-{4-[4-(3,4-Dichloro- | 457/459 | 0.93 (6H, t), 1.09-1.20 (1H, |
| | phenoxy)-piperidin-1- | | m), 1.29 (2H, t), |
| | ylmethyl]-piperidin-1-yl}-4- | | 1.49-1.67 (2H, m), 1.69-1.81 (5H, m), |
| | methyl-pentanoic acid | | 1.93-2.02 (2H, m), 2.20 (2H, |
| | | | d), 2.24-2.40 (4H, m), |
| | | | 2.64-2.74 (2H, m), 2.95 (2H, d), |
| | | | 3.00-3.06 (1H, m), |
| | | | 4.31-4.41 (1H, m), 6.85-6.89 (1H, |
| | | | m), 7.07 (1H, d), 7.36 (1H, d) |
| 11A | 2-{4-[4-(4-Chloro-2-methyl- | 423/425 | 1.01 (3H, d), 1.13 (3H, d), |
| | phenoxy)-piperidin-1- | | 1.41-1.60 (2H, m), |
| | ylmethyl]-piperidin-1-yl}-3- | | 1.75-1.91 (4H, m), 1.93-2.04 (6H, m), |
| | methyl-butyric acid | | 2.16 (3H, s), 2.31 (2H, d), |
| | | | 2.36-2.44 (1H, m), |
| | | | 2.68-2.76 (2H, m), 2.93-3.05 (2H, m), |
| | | | 3.45-3.56 (2H, m), |
| | | | 4.35-4.42 (1H, m), 6.86 (1H, d), |
| | | | 7.06 (1H, d), 7.09 (1H, d) |

### EXAMPLE 12A

### 1-{4-[4-(4-Fluoro-2-methyl-phenoxy)-piperidin-1-ylmethyl]-piperidin-1-yl}-cyclohexanecarboxylic acid dihydrochloride

tert-Butyl 1-{4-[4-(4-fluoro-2-methyl-phenoxy)-piperidin-1-ylmethyl]-piperidin-1-yl}-cyclohexanecarboxylate (0.2 g) was stirred and sonicated in aq. HCl (20 mL, 6M) for 16 h. The solvents were evaporated and the residue was redissolved in aqueous ammonium acetate solution; acetonitrile was added. The layers were separated and the organic layer was evaporated, a solid formed which was filtered, washed with water and ether. The solid was taken up in 6M aq. HCl and evaporated to give the title compound (64 mg).

¹H NMR δ_{(CD3OD+NaOD)} 1.11-1.83 (15H, m), 1.92-2.01 (2H, m), 2.13-2.35 (11H, m), 2.63-2.71 (2H, m), 3.08-3.16 (2H, m), 4.25-4.34 (1H, m), 6.75-6.90 (3H, m)

MS [M+H]+ 433 (ES+)

RT 1.23 (standard)

### EXAMPLE 13A

### 1-(4-{[4-(4-Chloro-2-methylphenoxy)piperidin-1-yl]methyl}piperidin-1-yl)cyclohexanecarboxylic acid dihydrochloride

tert-Butyl 1-{4-[4-(4-chloro-2-methyl-phenoxy)-piperidin-1-ylmethyl]-piperidin-1-yl}-cyclohexanecarboxylate (0.18 g) was stirred in 6 M HCl (10 mL) for 16 h. Additional HCl (conc, 3 mL) was added and the mixture was stirred for a further 3 h. The volume of solvent was reduced and product precipitated to give the title compound (24 mg).

¹H NMR δ_{(CD3OD+NaOD)} 1.18-1.35 (1H, m), 1.42-1.59 (2H, m), 1.66-1.79 (5H, m), 1.81-1.95 (2H, m), 1.95-2.12 (1H, m), 2.14-2.37 (10H, m), 2.41-2.50 (2H, m), 3.10-3.22 (6H, m), 3.50-3.58 (1H, m), 3.65-3.78 (3H, m), 6.90-6.99 (1H, m), 7.10-7.20 (2H, m)

MS [M+H]+ 449/451 (APCI+)

RT 1.70 (std)

### EXAMPLE 14A

### (2S)-2-(4-{[4-(3,4-Dichlorophenoxy)piperidin-1-yl]methyl}piperidin-1-yl)-3-methylbutanoic acid

Methyl (2S)-2-(4-{[4-(3,4-dichlorophenoxy)piperidin-1-yl]methyl}piperidin-1-yl)-3-methylbutanoate (35 mg) was taken up in aq. HCl (6 M, 40 mL)) and the reaction mixture was heated to 80° C. for 48 h. The solvents were evaporated, the residue was taken up in methanol and purified via RP-prep-HPLC (gradient 0.1% aqueous ammonium acetate:acetonitrile 95:5 to 50:50 over 25 min) to give title compound (22 mg).

¹H NMR δ_{(CD3OD+NaOD)} 1.02 (3H, d), 1.14 (3H, d), 1.26-1.38 (3H, m), 1.42-1.64 (2H, m), 1.73-1.85 (2H, m), 1.98-2.07 (4H, m), 2.28-2.39 (2H, m), 2.40-2.49 (2H, m), 2.75-2.84 (2H, m), 2.97-3.04 (2H, m), 3.46-3.58 (2H, m), 4.39-4.46 (1H, m), 6.89 (1H, dd), 7.10 (1H, d), 7.38 (1H, d)

MS [M+H]+ 443/445 (APCI+)

RT 1.58 (std)

### EXAMPLE 15A

### (2S)-2-{4-[4-(3,4-Dichloro-2-methyl-phenoxy)-piperidin-1-ylmethyl]-piperidin-1-yl}-2,4-dimethyl-pentanoic acid

A mixture of methyl (2S)-2-{4-[4-(3,4-Dichloro-2-methyl-phenoxy)-piperidin-1-ylmethyl]-piperidin-1-yl}-2,4-dimethyl-pentanoate (50 mg), barium hydroxide (82 mg), NMP (2 mL), water (1 mL) and methanol (1 mL) were heated together in a microwave at 190° C. for 3 h. The mixture was then acidified with acetic acid (1 mL), filtered, and purified by reverse-phase hplc (95:5 0.1% aqueous ammonium acetate/acetonitrile to 5:95 0.1% aqueous ammonium acetate/acetonitrile over 10 minutes, symmetry column) to give the title compound (33 mg).

¹H NMR ∂_{(CD3OD+NaOD)} 0.89-0.94 (6H, m), 1.15-1.30 (3H, m), 1.17 (3H, s), 1.39-1.45 (1H, m), 1.46-1.57 (1H, m), 1.64-1.84 (5H, m), 1.94-2.03 (2H, m), 2.08 (2H, t), 2.21 (2H, d), 2.27-2.37 (2H, m), 2.30 (3H, s), 2.61-2.71 (2H, m), 2.95 (1H, d), 3.04 (1H, d), 4.36-4.45 (1H, m), 6.90 (1H, d), 7.26 (1H, d)

MS [M+H]+ 485/487 (APCI+)

The following compound was prepared by the method of Example 15A:

| | | MS [M + H]⁺ | |
|---|---|---|---|
| | | (APCI+); | |
| Example | Name | RT (std) | ¹H NMR |
| 16A | (2S)-2-{4-[4-(4-Chloro-2- | 449/451 | 0.95-1.06 (6H, m), |
| | methyl-phenoxy)-piperidin-1- | | 1.29-1.37 (1H, m), 1.39-1.48 (3H, |
| | ylmethyl]-piperidin-1-yl}-2,4- | | m), 1.52-1.70 (3H, m), |
| | dimethyl-pentanoic acid | | 1.74-1.90 (5H, m), 1.95-2.13 (4H, |
| | | | m), 2.20 (3H, s), 2.30 (2H, d), |
| | | | 2.34-2.46 (2H, m), |
| | | | 2.66-2.79 (2H, m), 2.88-3.13 (3H, |
| | | | m), 4.35-4.48 (1H, m), |
| | | | 6.90 (1H, d), 7.05-7.17 (2H, m) |

### EXAMPLE 17

### Human Eosinophil Chemotaxis

Human eosinophils are isolated from EDTA anticoagulated peripheral blood as previously described (Hansel et al., J. Immunol. Methods, 1991, 145, 105-110). The cells are resuspended at 10×10⁶ mL⁻¹ in RPMI containing 200 IU/mL penicillin, 200 μg/mL streptomycin sulfate and supplemented with 10% HIFCS, at room temperature.

Eosinophils (700 μl) ae pre-incubated for 15 mins at 37° C. with 7 μl of either vehicle or compound (100× required final concentration in 10% DMSO). A chemotaxis plate (ChemoTx, 3 μm pore, Neuroprobe) can be loaded by adding 28 μl of a concentration of eotaxin 0.1 to 100 nM (a selective CCR3 agonist over this concentration range) containing a concentration of a compound according to the Examples or solvent to the lower wells of the chemotaxis plate. The filter is then placed over the wells and 25 μl of eosinophil suspension is added to the top of the filter. The plate is incubated for 1 hr at 37° C. in a humidified incubator with a 95% air/5% CO₂ atmosphere to allow chemotaxis.

The medium, containing cells that had not migrated, is carefully aspirated from above the filter and discarded. The filter is then washed once with phosphate buffered saline (PBS) containing 5 mM EDTA to remove any adherent cells. Cells that have migrated through the filter are pelleted by centrifugation (300×g for 5 mins at room temperature) and the filter removed and the supernatant transferred to each well of a 96-well plate (Costar). The pelleted cells are lysed by the addition of 28 μl of PBS containing 0.5% Triton ×100 followed by two cycles of freeze/thawing. The cell lysate is then added to the supernatant. The number of eosinophils migrating can be quantified according to the method of Strath et al., J. Immunol. Methods, 1985, 83, 209 by measuring eosinophil peroxidase activity in the supernatant.

### EXAMPLE 18

Histamine H1 receptor binding activity of compounds of the invention was assessed by competition displacement of 1 nM [3H]-pyrilamine (Amersham, Bucks, Product code TRK 608, specific activity 30 Ci/mmol) to 2 μg membranes prepared from recombinant CHO-K1 cells expressing the human H1 receptor (Euroscreen SA, Brussels, Belgium, product code ES-390-M) in assay buffer (50 mM Tris pH 7.4 containing 2 mM MgCl₂, 250 mM sucrose and 100 mM NaCl) for 1 hour at room temperature.

The following compounds of the invention gave inhibition of [3H]pyrilamine binding:

| Example | H1 pKi |
|---|---|
| 1A | 7.2 |
| 2A | 7.1 |
| 3A | 6.7 |
| 4A | 6.9 |
| 6A | 6.3 |
| 8A | 6.5 |
| 13A | 6.2 |

### EXAMPLE 19

### Eotaxin-2-Induced Shape Change in Eosinophils in Human Blood In Vitro

See for example, Differential regulation of eosinophil chemokine signaling via CCR3 and non-CCR3 pathways. Sabroe I, Hartnell A, Jopling L A, Bel S, Ponath P D, Pease J E, Collins P D, Williams T J. J Immunol. 1999 Mar. 1; 162(5):2946-55.

Human blood, collected by venous puncture into 9 mL lithium-heparin tubes, was incubated with the CCR3 agonist eotaxin-2 in the presence of vehicle (0.1% (v/v) DMSO) or test compound for 4 min at 37° C. in a deep, 96-square-well plate. The blood was fixed with Optilyse B (100 μL) at room temperature for 10 min and then the red blood cells were lysed with distilled water (1 mL) for 60 min at room temperature.

The plate was centrifuged at room temperature for 5 min at 300 g. The pellet was re-suspended in assay buffer (PBS without CaCl₂ and MgCl₂, containing HEPES (10 mM), Glucose (10 mM) and 0.1% (w/v) BSA, pH 7.4)) and the samples were analysed using flow cytometry (FC500, Beckman Coulter). The high autofluorescence of eosinophils allowed them to be identified as a discrete population from the other blood cell types. Eosinophil shape was monitored as the refractive index of the eosinophil population as determined using the forward scatter signal in flow cytometry.

Eotaxin-2 induced a concentration-dependent change in the forward scatter of eosinophils and these data were used to construct a concentration effect curve (E/[A] curve). The rightward displacement of the eotaxin-2 E/[A] curve in the presence of a CCR3 antagonist was used to estimate a pA₂ value in blood using the following equation:

Single pA₂=−log₁₀([B]/(r−1))

where r is the ratio of the concentrations required for half maximal effects of eotaxin-2 in the absence and presence of antagonist ([A]₅₀ for eotaxin-2 in the presence of antagonist divided by [A]₅₀ for control eotaxin-2 curve) and [B] is the molar concentration of antagonist.

### EXAMPLE 20

### Determination of Compound Affinity at Human Recombinant CCR3 Receptors Assessed by Competition of [³H]-4-(2,4-dichloro-3-methylphenoxy)-1′-[4-(methylsulfonyl)benzoyl]-1,4′-bipiperidine for CHO-K1 Cell Membranes In Vitro

Membranes, prepared from CHO-K1 cells stably expressing recombinant human CCR3, suspended in assay buffer (50 mM Tris-Base, pH 7.4; containing sodium chloride (100 mM) and magnesium chloride (2 mM)) were incubated in the presence of 2 nM [³H]-4-(2,4-dichloro-3-methylphenoxy)-1′-[4-(methylsulfonyl)benzoyl]-1,4′-bipiperidine, along with vehicle (1% (v/v) DMSO), 4-(4-chloro-3-methylphenoxy)-1′-[2-(methylsulfonyl)benzoyl]-1,4′-bipiperidine (to define non-specific binding) or test compound for 2 h at 37° C. in round bottomed 96-well plates. The plates were then filtered onto GF/B filter plates, pre-soaked for 1 hour in plate-coating solution (0.3% (w/v) polyethylenimine, 0.2% (w/v) BSA in de-ionised water), using a 96-well plate Tomtec cell harvester. Four washes (250 μL) with wash buffer (50 mM Tris-Base, pH 7.4 containing sodium chloride (500 mM) and magnesium chloride (2 mM)) were performed at 4° C. to remove unbound radioactivity. Plates were dried and MicroScint-O (50 μL) was added to each well. The plates were sealed (TopSeal A) and filter-bound radioactivity was measured with a scintillation counter (TopCount, Packard BioScience) using a 1 minute counting protocol.

Specific binding was determined from values of the control wells minus the values for the NSB wells for each assay plate. pIC₅₀ values were calculated using a four parameter logistic fit (where pIC₅₀ is defined as the negative logarithm of the concentration of compound required for 50% reduction in specific [³H]-4-(2,4-dichloro-3-methylphenoxy)-1′-[4-(methylsulfonyl)benzoyl]-1,4′-bipiperidine binding). Data were presented as mean pKi values (calculated by applying a Cheng-Prussof correction to pIC₅₀ values) from a minimum of 2 separate experiments.

The following compound of the invention gave inhibition of binding:

| Example | CCR3 pKi |
|---|---|
| 16A | 9.2 |

1. A compound of formula (I): 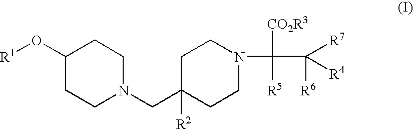
wherein:
R¹ is phenyl optionally substituted by halogen, cyano, C₁₋₄ alkyl or C₁₋₄ alkoxy;
R² is hydrogen or hydroxy;
R³ is hydrogen, C₁₋₆ alkyl or phenyl(C₁₋₄ alkyl); wherein phenyl is optionally substituted with halogen, hydroxy, nitro, S(O)_{q}(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
q is 0, 1 or 2;
R⁴ is methyl, CH(CH₃)₂, or C₃₋₇ cycloalkyl optionally substituted by C₁₋₄ alkyl;
R⁵, R⁶ and R⁷ are, independently, hydrogen or methyl;
or R⁴ and R⁵ join to form a 3-7 membered carbocyclic ring optionally substituted by C₁₋₄ alkyl; and two of the ring carbons of this ring can be joined through a 1 or 2 carbon alkylene chain (which is itself optionally substituted by C₁₋₄ alkyl) such that a bicyclic ring system is formed;
or a N-oxide thereof; or a pharmaceutically acceptable salt thereof.

2. A compound as claimed in claim 1 wherein R¹ is phenyl optionally substituted with fluorine, chlorine, cyano or C₁₋₄ alkyl.

3. A compound as claimed in claim 1 wherein R² is hydrogen.

4. A compound as claimed in claim 1 wherein R³ is hydrogen or C₁₋₆ alkyl.

5. A compound as claimed in claim 1 wherein R³ is hydrogen.

6. A compound as claimed in claim 1 that is a sodium or potassium salt of a compound of formula (I) wherein R³ is hydrogen.

7. A compound as claimed in claim 1 wherein R⁴ is CH(CH₃)₂.

8. A compound as claimed in claim 1 wherein R⁵ is hydrogen.

9. A compound as claimed in claim 1 wherein R⁵ is methyl.

10. A compound as claimed in claim 1 wherein R⁶ and R⁷ are both hydrogen.

11. A process for preparing a compound as claimed in claim 1, the process comprising:
a. reacting a compound of formula (II): 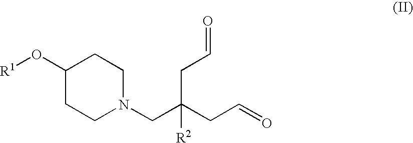
with a compound of formula (III): 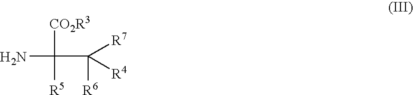
in the presence of NaBH(OAc)₃ or NaBH₃(CN) in a suitable solvent at a suitable temperature;
b. when R is alkyl or phenylalkyl, reacting a compound of formula (II) with a compound of formula (III), where R³ is alkyl or phenylalkyl, in the presence of NaBH(OAc)₃ in the presence of a suitable base, in a suitable solvent, at a suitable temperature;
c. when R³ is hydrogen said compound may be converted to a compound of the invention where R³ is not hydrogen by a standard esterification or salt formation method well known in the art; or
d. when R³ is not hydrogen said compound may be converted to a compound of the invention where R³ is hydrogen by a standard ester hydrolysis or acidification method well known in the art.

12. A pharmaceutical composition which comprises a compound of the formula (I), or a pharmaceutically acceptable salt thereof as claimed in claim 1, and a pharmaceutically acceptable adjuvant, diluent or carrier.

13-14. (canceled)

15. A method of treating a chemokine mediated disease state in a mammal suffering from, or at risk of, said disease, which comprises administering to a mammal in need of such treatment a therapeutically effective amount of a compound of formula (I), or a pharmaceutically acceptable salt thereof as claimed in claim 1.
